(19) United States
(12) Patent Application Publication
(10) Pub. No.: US 2011/0029095 A1
(43) Pub. Date: Feb. 3, 2011

(54) HIP PROSTHESIS

(76) Inventors: Jean-Claude CARTILLIER, Lyon (FR); Laurent JACQUOT, Charvonnex (FR); Bruno BALAY, Trevoux (FR); Claude CHARLET, Saint Didier Au Mont D'or (FR); Michel Henri Fessy, Saint Genis Laval (FR); Jean Marc SEMAY, St. Priest en Jarez (FR); Louis SETIEY, Gleize (FR); Jean-Christophe CHATELET, Jassans (FR); Jean Pierre Vidalain, Annecy Le Vieux (FR); Alain MACHENAUD, La Balme De Sillingy (FR); Tarik AIT SI SELMI, Lyon (FR); Jean-Charles ROLLIER, Saint Martin Bellevue (FR)

Correspondence Address: CANTOR COLBURN LLP, 20 Church Street, 22nd Floor, Hartford, CT, 06103, US

(21) Appl. No.: 12/817,737

(22) Filed: Jun. 17, 2010

(30) Foreign Application Priority Data
Jun. 18, 2009 (FR) 0954092

(51) Int. Cl. A61F 2/32

(52) U.S. Cl. 623/22.46

(57) ABSTRACT

Hip prosthesis including a medullar part of elongated shape intended to be engaged in the medullary canal of a femur, one part forming the prosthetic femoral neck intended to project from the femur and be fitted with a prosthetic femoral head, and a support collar extending transversely to the part forming the femoral neck and intended to take support on the femoral cortex, where the support collar includes a slot.

### TECHNICAL FIELD

This invention relates to a hip prosthesis.

### BACKGROUND

A hip prosthesis comprises, in a known manner, an elongated shape medullar part intended to be engaged in the medullary canal of a femur, a part forming a prosthetic femoral neck intended to project from the femur and to be fitted with a prosthetic femoral head and a support collar extending transversally to the part forming the femoral neck and intended to take support on the femoral cortex, and more particularly on the spur of Meckel, also called calcar.

The presence of the collar support allows on one hand to prevent a penetration of the medullar part in the medullar canal of the patient beyond a specified area, and on the other hand to improve the seat of the prosthesis.

However, such a collar support has the disadvantage of causing a peak of stresses in the calcar, which may generate a bone degradation of the latter.

In addition, since such a collar support covers the internal cortex of the femur and part of the front and rear cortices of the latter, it prevents access from the top to the internal, front and rear surfaces of the medullar part with osteotomes. Thus, when it is desired to extract the hip prosthesis, it is necessary to perform a resection of the femur below the collar support in order to be able to reach the internal, front and rear surfaces of the medullar part with osteotomes intended to dissociate the medullar part of the femur. This results in a deterioration of the femur bone and complex surgery.

### BRIEF SUMMARY

The purpose of this invention is to overcome these drawbacks.

The invention therefore seeks to provide a hip prosthesis that is simple in structure and economic, and that allows to simplify and improve surgery, while limiting the constraints applied on the calcar.

To this end, the invention relates to a hip prosthesis comprising an elongated medullar part intended to be engaged in the medullary canal of a femur, one part forming prosthetic femoral neck intended to project from the femur and be equipped with prosthetic femoral head and a collar support extending transversally to the part forming the femoral neck and intended to take support on the femoral cortex, the collar support comprising at least a medial portion projecting from the inner surface of the medullar part and lateral portions projecting respectively from the front and rear medullar part, characterised in that the collar support has a slot extending along the medial portion of the collar support and also on the lateral portions of the collar support.

The presence of a slot on the collar support allows viewing the effective support of the latter on the femoral cortex, and hence to ensure a perfect positioning of the medullar part.

The presence of such a slot on the collar support allows furthermore a visual check of the possible presence of cracks or fractures of the calcar after implementation of the medullar part, and therefore to make the necessary arrangements as soon as possible (for example make a cerclage of the femur) to allow a rapid consolidation of the crack or fracture.

Moreover, the presence of such a slot on the collar support makes the latter more flexible, which allows to modify the constraints applied by the collar support on the calcar, and in particular to avoid peak constraints on the latter.

The fact that the slot extends on the medial portion of the collar support and furthermore on the side portions of the latter allows access from the top to the internal, front and rear surfaces of the medullar part with osteotomes. The result is a simplified surgical operation in case of relapse of the hip prosthesis.

Conveniently, the slot is arranged to allow passage of osteotomes, such as osteotomy blades, for example flat, curved or gouge shaped.

Preferably, the underside of the collar support is coated with a calcium phosphate coating such as hydroxyapatite and tricalcium phosphate.

In a preferable manner, the slot extends substantially perpendicular to the underside of the collar support.

Preferably, the slot has a substantially kidney shape.

According to a characteristic of the invention, one of the edges of the slot extends into the continuity of the inner surface of the medullar part. These provisions allow a guide of the osteotomes along the slot, and preventing the latter to abut against the inner surface of the medullar part and against the edge of the calcar.

Conveniently, the prosthesis comprises a removable shutter arranged to shut the slot. Such a shutter is designed to seal off the slot after insertion of the medullar part in the medullary canal of the femur and checking that the latter is perfectly positioned and that there are no cracks in the calcar. These provisions allow to prevent migration of wear particles from the prosthesis towards the interface medulla-femur, and hence the creation of osteolysis that may cause loosening of the prosthesis. These provisions allow also forming an obstacle to femoral bleeding.

According to another characteristic of the invention, the collar support is removable. Preferably, the collar support has the shape of a U-shaped bracket comprising two secure shutter arms at each end of a central part, the shutting branches being intended to be engaged respectively in complementary grooves formed in the front and rear surfaces of the medullar part, and the central part being intended to abut against the inner surface of the medullar part

Conveniently, the collar support is located at the junction between the medullar part and the part forming the femoral neck.

According to an embodiment of the invention, the hip prosthesis comprises at least a wedge intended to be positioned below the collar support in order to be interposed between the latter and the femur, the wedge comprising fixing means arranged to cooperate with the slot formed on the collar support.

According to an embodiment of the invention, the hip prosthesis comprises a distribution device for at least one drug substance, such as an antibiotic, intended to be mounted in the slot formed on the collar support. The fact of positioning such a distribution device in the slot formed in the collar support allows distributing locally a drug substance, which prevents injecting intravenously a large dose of drug substance to the patient. Moreover, this positioning of the distribution device in the slot ensures the distribution of a drug substance on the one hand towards the medullar canal and on the other hand towards the joint cavity.

The distribution device comprises conveniently a portion having a shape at least partially complementary to the slot. The distribution device may be, for example, a solid tablet or capsule, preferably resorbable, enclosing at least one drug substance.

### BRIEF DESCRIPTION OF THE DRAWINGS

In any case, the invention will be better understood from the following description with reference to the attached schematic drawing representing, by way of a non limiting example, an embodiment of this hip prosthesis.

FIG. 1 is a perspective partial view of a hip prosthesis according to the invention.

FIG. 2 is a profile partial view of the prosthesis of FIG. 1.

FIG. 3 is a perspective view of a wedge according to a first embodiment of the invention.

FIG. 4 is a perspective view of a wedge according to a second embodiment of the invention.

FIG. 5 is a perspective view of a distribution device according to the invention.

### DETAILED DESCRIPTION

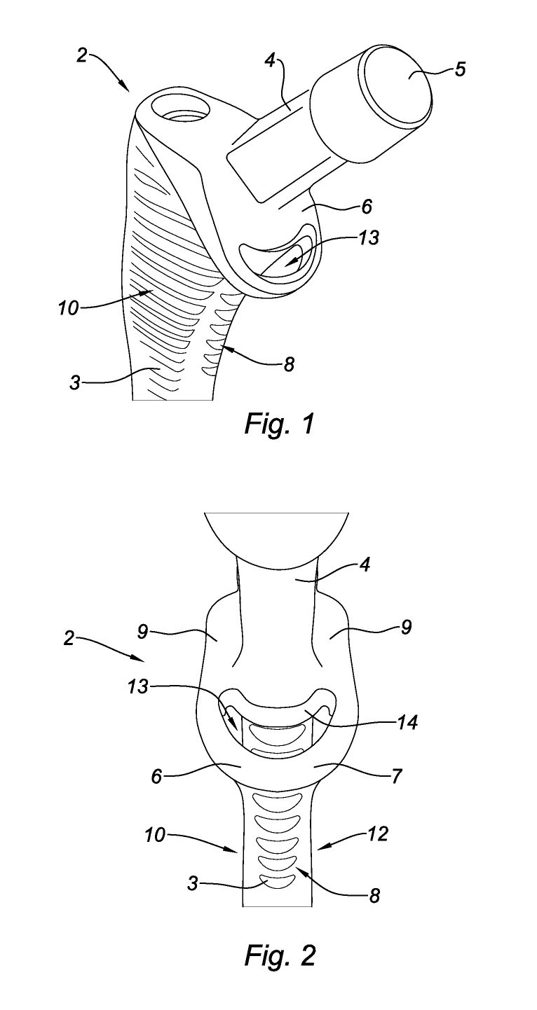

FIGS. 1 and 2 represent a hip prosthesis 2 preferably made of titanium, titanium alloy or chrome cobalt.

The hip prosthesis 2 comprises a medullar part 3 of elongated shape intended to be engaged in the medullary canal of a femur, and a part forming a prosthetic femoral neck 4 intended to project from the femur and be fitted with a prosthetic femoral head (not shown in the Figures).

The part forming the femoral neck 4 comprises a tapered proximal end 5 arranged to cooperate with a tapered cavity of complementary shape formed in the prosthetic femoral head.

The hip prosthesis 2 also comprises a collar support 6 located at the junction between the medullar part 3 and the part forming the femoral neck 4.

The support collar 6 extends substantially perpendicular to the part forming the femoral neck 4. The collar support 6 extends also substantially at an angle of about 45° in relation to the dialphyseal axis.

The support collar 6 is intended to take support on the inner cortex of the femur after section of the latter and depressing the medullar part 3 in the medullar canal. The support collar 6 is more particularly intended to take support on the spur of Merckel, also called calcar.

As shown more particularly in FIG. 2, the collar support 6 comprises a medial portion 7 projecting from the inner surface 8 of the medullar part 3 and side portions 9 projecting from the front 10 and rear 12 surfaces respectively of the medullar part 3. It should be noted that the inner surface 8 of the medullar part 3 is intended to be turned towards the calcar, while the outer surface thereof is intended to be turned towards the greater trochanter, the front and rear surfaces connecting the inner and outer surfaces of the medullar part 3.

The collar support 6 comprises a slot 13 having a kidney shape. Slot 13 extends on the medial portion 7 and the lateral portions 9 of the collar support 6. The length of the slot 13 according to an anteroposterior plane is preferably comprised between 3 and 5 mm.

The slot 13 extends substantially perpendicularly to the underside of the collar support 6. The edge 14 of the slot 6 located near the part forming the femoral neck portion 4 extends into the continuity of the inner surface 8 of the medullar part 3.

The slot 13 is arranged to allow passage of osteotomes, such as osteotomy flexible blades.

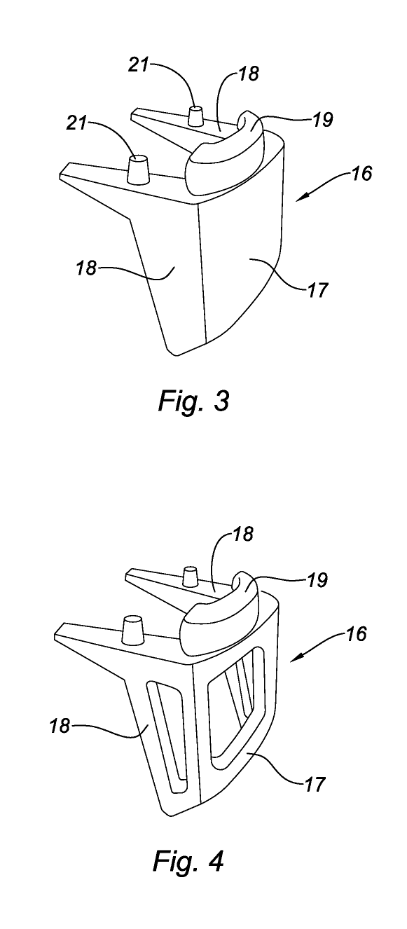

As shown in FIG. 3, the hip prosthesis also comprises furthermore a wedge 16 intended to be positioned below the collar support 6 in order to be interposed between the latter and the femur. The wedge 16 allows to compensate for bone losses, in particular the calcar, and hence to rebuild support for the hip prosthesis. The wedge 16 is preferably made of titanium.

The wedge 16 has the shape of a U bracket comprising a central portion 17 intended to extend beneath the medial portion 7 of the collar support 6, and two lateral branches 18 secured to the central portion 17 and intended to be extended respectively under the side portions 9 of the collar support 6.

The central portion 17 has a profile being complementary to that of the inner surface 8 of the medullar part 3 and is intended to abut against the inner surface 8 of the latter.

To ensure fixation of the wedge 16 on collar support 6, the wedge 16 comprises on one hand a member 19 projecting from the upper surface of the central portion 17 of the wedge and arranged to engage in slot 13, and on the other hand two lugs 21 projecting respectively from the upper surface of each lateral branch 18 of the wedge and arranged to cooperate with two additional holes (not shown in the Figures) provided respectively on the undersides of the lateral portions 9 of the collar support 6.

According to an embodiment shown in FIG. 4, the lateral branches 18 and the central portion 17 of the wedge 16 are perforated to promote bone growth and also to allow the attachment of bone graft or bone substitute.

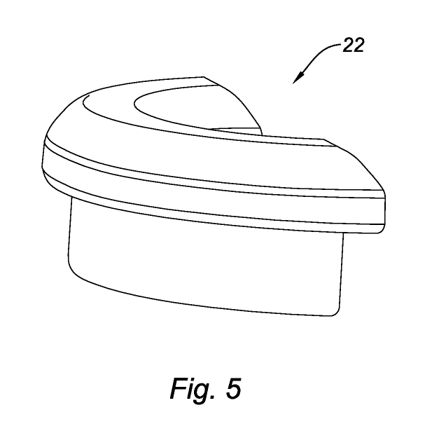

As shown in FIG. 5, the hip prosthesis also comprises a distribution device 22 of at least a drug substance, such as an antibiotic, intended to be mounted on the slot 13 provided on the collar support. The distribution device 22 comprises a portion 23 having a shape at least partially complementary to slot 13.

The distribution device may be, for example, a solid tablet or capsule, preferably resorbable, enclosing one or more drug substances.

According to an embodiment of the invention, the collar support 6 is removable. According to this embodiment, the collar support has the shape of a U-shaped bracket comprising two secured shutting branches to each end of a central part. The shutting branches of the collar support are intended to be respectively engaged in complementary grooves formed in the front and rear surfaces of the medullar part, each groove being inclined upwards, and the internal surface towards the external surface of the medullar part. The central part of the collar support is intended to abut against the inner surface of the medullar part.

According to an embodiment of the invention not shown in the Figures, the hip prosthesis comprises a removable shutter arranged to seal off the slot formed on the collar support.

According to another embodiment of the invention not shown in the Figures, the medullar part and the part forming the femoral neck may not be integral. Accordingly, the section forming the femoral neck could be removable and include a substantially cylindrical central portion, a tapered distal end arranged to cooperate with a tapered cavity of complementary shape formed in the proximal end of the medullar part, and a tapered proximal end arranged to cooperate with a tapered cavity of complementary shape formed in the prosthetic femoral head.

It is understood that the invention is not limited to the sole embodiment of this hip prosthesis, as described above as a non-limiting example; on the contrary, it encompasses all alternative embodiments. Thus, in particular the hip prosthesis according to the invention may be a hip prosthesis by biological fixation or by cemented fixation.

1. Hip prosthesis comprising:
an elongated shape medullar part intended to be engaged in a medullary canal of a femur,
a part forming a prosthetic femoral neck intended to project from the femur and to be fitted with a prosthetic femoral head, and
a support collar extending transversally to the part forming the femoral neck and intended to take support on a femoral cortex,
wherein the support collar comprises a medial portion projecting from an inner surface of the medullar part and lateral portions projecting respectively from front and rear surfaces of the medullar part, and
wherein the support collar further comprises a slot extending over the medial portion of the support collar and furthermore on the lateral portions of the support collar.

2. Prosthesis according to claim 1, wherein the slot extends substantially perpendicularly to an underside of the support collar.

3. Prosthesis according to claim 1, wherein the slot has a substantially kidney shape.

4. Prosthesis according to claim 1, wherein one edge of the slot extends in continuity with the inner surface of the medullar part.

5. Prosthesis according to claim 1, wherein the support collar is removable.

6. Prosthesis according to claim 5, wherein the support collar has a shape of a U-shaped bracket comprising two shutting branches secured to each end of a central part, the shutting branches being intended to be engaged respectively in complementary grooves formed in the front and rear surfaces of the medullar part, and the central part being intended to abut against the inner surface of the medullar part.

7. Prosthesis according to claim 1, further comprising a removable shutting engaged to shut the slot.

8. Prosthesis according to claim 1, further comprising at least one wedge intended to be positioned under the collar support in order to be interposed between the latter and the femur, the wedge comprising fixing means engaged to cooperate with the slot formed on the collar support.

9. Prosthesis according to claim 1, further comprising a distribution device of at least a drug substance intended to be mounted on the slot formed on the collar support.

10. Prosthesis according to claim 9, wherein the distribution device comprises a portion having a shape at least partially complementary of the slot.

11. Prosthesis according to claim 9, wherein the distribution device is a solid tablet or capsule containing a drug substance.
